(19) United States
(12) Patent Application Publication
(10) Pub. No.: US 2005/0228300 A1
(43) Pub. Date: Oct. 13, 2005

(54) Cuffless blood-pressure monitor and accompanying wireless mobile device

(75) Inventors: Manuel Jaime, Solana Beach, CA (US); Henk Visser, San Diego, CA (US); Matthew J. Banet, Del Mar, CA (US); Brett Morris, San Diego, CA (US)

Correspondence Address: MATTHEW J. BANET, 6540 LUSK BLVD., C200, SAN DIEGO, CA, 92121, US

(73) Assignee: TRIAGE DATA NETWORKS

(21) Appl. No.: 10/967,511

(22) Filed: Oct. 18, 2004

Related U.S. Application Data
(63) Continuation-in-part of application No. 10/709,014, filed on Apr. 7, 2004.

(51) Int. Cl. A61B 5/02

(52) U.S. Cl. 600/485; 128/903

(57) ABSTRACT

The present invention provides a system for monitoring blood pressure that preferably includes: 1) a blood-pressure monitor featuring a measuring component that generates blood-pressure information and a first short-range wireless component configured to wirelessly transmit the blood-pressure information; 2) a mobile device featuring a chipset that includes i) an embedded second short-range wireless component configured to receive the blood-pressure information; and ii) a long-range wireless component configured to transmit the blood-pressure information over a wireless network; and 3) a computer system configured to receive and display the blood-pressure information.

### CROSS REFERENCES TO RELATED APPLICATION

This application is a continuation-in-part application of U.S. patent application Ser. No. 10/709,014, filed on Apr. 7, 2004.

### STATEMENT REGARDING FEDERALLY SPONSORED RESEARCH OR DEVELOPMENT

Not Applicable

### BACKGROUND OF THE INVENTION

1. Field of the Invention

The present invention relates to a system that measures blood-pressure information.

2. Description of Related Art

Blood within a patient's body is characterized by a baseline pressure value, called the diastolic pressure. Diastolic pressure indicates the pressure in an artery when the blood it contains is static. A heartbeat forces a time-dependent volume of blood through the artery, causing the baseline pressure to increase in a pulse-like manner to a value called the systolic pressure. The systolic pressure indicates a maximum pressure in a portion of the artery that contains a flowing volume of blood. Pressure in the artery periodically increases from the diastolic pressure to the systolic pressure in a pulsatile manner, with each pulse corresponding to a single heartbeat. Blood pressure then returns to the diastolic pressure when the flowing pulse of blood passes through the artery.

Both invasive and non-invasive devices can measure a patient's systolic and diastolic blood pressure. A non-invasive medical device called a sphygmomanometer measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting periodic acoustic ‘beats’ or ‘taps’ indicating that the blood is flowing past the cuff (i.e., the systolic pressure barely exceeds the cuff pressure). The minimum pressure in the cuff that restricts blood flow is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Low-cost, automated devices measure blood pressure using an inflatable cuff and an automated acoustic or pressure sensor that measures blood flow. These devices typically feature cuffs fitted to measure blood pressure in a patient's wrist, arm or finger. During a measurement, the cuff automatically inflates and then incrementally deflates while sensing electronics (located in the cuff or in an external device) measure changes in pressure and consequently blood flow. A microcontroller in the external device then processes this information to determine blood pressure. Cuff-based blood-pressure measurements such as these typically only determine the systolic and diastolic blood pressures; they do not measure dynamic, time-dependent blood pressure.

Time-dependent blood pressure can be measured with a device called a tonometer. The tonometer features a sensitive transducer positioned on the patient's skin above an underlying artery. The tonometer compresses the artery against a portion of bone, during which time the transducer measures blood pressure in the form of a time-dependent waveform. The waveform features a baseline that indicates the diastolic pressure, and time-dependent pulses, each corresponding to individual heartbeats. The maximum value of each pulse is the systolic pressure. The rising and falling edges of each pulse correspond to pressure values that lie between the systolic and diastolic pressures.

Data indicating blood pressure are most accurately measured during a patient's appointment with a medical professional, such as a doctor or a nurse. Once measured, the medical professional manually records these data in either a written or electronic file. Appointments typically take place a few times each year. Unfortunately, patients often experience ‘white coat syndrome’ where anxiety during the appointment affects the blood pressure that is measured. For example, white coat syndrome can elevate a patient's heart rate and blood pressure; this, in turn, can lead to an inaccurate diagnosis.

Pulse oximeters are devices that measure variations in a patient's arterial blood volume. These devices typically feature a light source that transmits optical radiation through the patient's finger to a photodetector. A processor in the pulse oximeter monitors time and wavelength-dependent variations in the transmitted radiation to determine heart rate and the degree of oxygen saturation in the patient's blood. Various methods have been disclosed for using pulse oximeters to obtain arterial blood pressure. One such method is disclosed in U.S. Pat. No. 5,140,990 to Jones et al., for a ‘Method Of Measuring Blood Pressure With a Photoplethysmograph’. The '990 patent discloses using a pulse oximeter with a calibrated auxiliary blood pressure to generate a constant that is specific to a patient's blood pressure. Another method for using a pulse oximeter to measure blood pressure is disclosed in U.S. Pat. No. 6,616,613 to Goodman for a ‘Physiological Signal Monitoring System’. The '613 patent discloses processing a pulse oximetry signal in combination with information from a calibrating device to determine a patient's blood pressure.

### BRIEF SUMMARY OF THE INVENTION

The present invention provides a cuffless, wrist-worn blood-pressure monitor that features a form factor similar to a conventional wristwatch. The blood pressure monitor makes a transdermal, optical measurement of blood pressure and wirelessly sends this information to a mobile device (e.g., a conventional cellular phone or PDA). The mobile device preferably features an embedded, short-range wireless transceiver and a software platform that displays, analyzes, and then transmits the information through a wireless network to an Internet-based system. With this system a medical professional can continuously monitor a patient's blood pressure during their day-to-day activities. Monitoring patients in this manner minimizes erroneous measurements due to ‘white coat syndrome’ and increases the accuracy of a blood-pressure measurement.

In one aspect, the invention provides a system for monitoring blood pressure that includes: 1) a blood-pressure monitor featuring a measuring component that generates blood-pressure information and a first short-range wireless component configured to wirelessly transmit the blood-pressure information; 2) a mobile device that includes i) an embedded second short-range wireless component configured to receive the blood-pressure information; and ii) a long-range wireless transceiver configured to transmit the blood-pressure information over a wireless network; and 3) a computer system configured to receive and display the blood-pressure information. For this system, ‘embedded’ means electronics for the short-range wireless component are integrated directly into the chipset, i.e. they are created during the microelectronic manufacturing of the chipset.

In another aspect, the invention provides a system for monitoring blood pressure that includes the above-mentioned system, with the embedded short-range wireless component replaced by a wireless component that connects to a serial port of a mobile device and features a second short-range wireless component configured to receive the blood-pressure information and send it to the mobile device.

The blood-pressure monitoring device typically features a short-range wireless transmitter operating on a wireless protocol that is matched to the wireless transceiver embedded in the mobile device. In typical embodiments the transceiver operates on a short-range wireless protocol such as Bluetooth™, 802.11a, 802.11b, 802.1g, or 802.15.4. A short-range wireless transmitter is defined as a transmitter capable of transmitting up to thirty meters. The mobile device also includes a long-range wireless transmitter that transmits information over a terrestrial wireless network, such as a network operating using a wireless protocol such as CDMA, GSM, GPRS, Mobitex, DataTac, iDEN, and analogs and derivatives thereof. A long-range wireless transmitter is defined as a transmitter capable of transmitting greater than thrity meters. Alternatively the network maybe based on a protocol such as 802.11a, 802.11b, 802.1g, or 802.15.4.

The invention has many advantages. In particular, it provides a system that continuously monitors a patient's blood pressure using a cuffless blood pressure monitor and an off-the-shelf mobile device. The mobile device can even be the patient's personal cellular phone. Information describing the blood pressure can be viewed using an Internet-based website, using a personal computer, or simply by viewing a display on the mobile device. Blood-pressure information measured continuously throughout the day provides a relatively comprehensive data set compared to that measured during isolated medical appointments. This approach identifies trends in a patient's blood pressure, such as a gradual increase or decrease, which may indicate a medical condition that requires treatment. The invention also minimizes effects of ‘white coat syndrome’ since the monitor automatically and continuously makes measurements away from a medical office with basically no discomfort to the patient. Real-time, automatic blood pressure measurements, followed by wireless transmission of the data, are only practical with a non-invasive, cuffless monitor like that of the present invention. Measurements can be made completely unobtrusive to the patient.

The monitor can also characterize the patient's heart rate and blood oxygen saturation using the same optical system for the blood-pressure measurement. This information can be wirelessly transmitted along with blood-pressure information and used to further diagnose the patient's cardiac condition.

The monitor is small, easily worn by the patient during periods of exercise or day-to-day activities, and makes a non-invasive blood-pressure measurement in a matter of seconds. The resulting information has many uses for patients, medical professional, insurance companies, pharmaceutical agencies conducting clinical trials, and organizations for home-health monitoring.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF SEVERAL VIEWS OF THE DRAWINGS

FIG. 1 is a semi-schematic view of a system according to the invention featuring a cuffless blood-pressure monitor that wirelessly relays blood-pressure information to a Bluetooth™-enabled mobile device, which in turn wirelessly transmits the information through a wireless network to a host and secondary computer systems;

FIG. 2 is a semi-schematic diagram showing ‘wired’ and ‘wireless’ methods for loading firmware applications into the mobile device of FIG. 1;

FIG. 3 is a schematic diagram of a firmware platform, operating on the mobile device of FIG. 1, for wirelessly receiving information from the blood-pressure monitor of FIG. 1;

FIG. 4 is a schematic diagram of the electrical components of the blood-pressure monitor of FIG. 1;

FIG. 5 is a schematic view of an Internet-based system, coupled with the system of FIG. 1, that transmits blood-pressure information through a wireless network to an Internet-accessible computer system;

FIGS. 6A and 6B are, respectively, front and side views of an alternative embodiment of the invention featuring a snap-on Bluetooth™-enabled device attached to a serial port on the bottom of mobile device of FIG. 1; and,

FIGS. 7A and 7B are side views of an alternative embodiment of the invention featuring a snap-on Bluetooth™-enabled device, respectively, separated and attached to a serial port on the back of mobile device of FIG. 1.

### DETAILED DESCRIPTION OF THE INVENTION

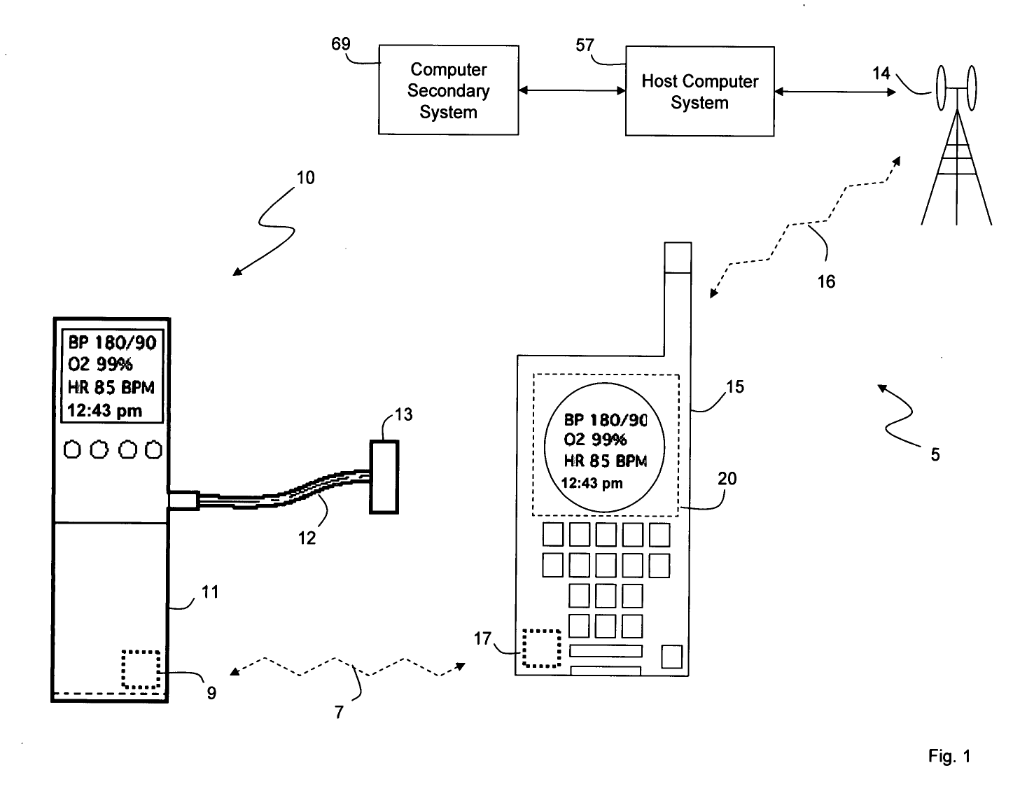

As shown in FIG. 1, a system 5 of the present invention preferably includes a cuffless blood-pressure monitor 10, a mobile device 15, a wireless network 14 and a computer 69. The cuffless blood-pressure monitor 10 preferably continuously measures a patient's real-time, beat-to-beat blood pressure. The monitor 10 preferably features an embedded Bluetooth™ transceiver 9 that sends information over a wireless link 7 to a matched transceiver 17 embedded in an “off-the-shelf” mobile device 15. The mobile device 15 includes a wireless transmitter 20 that wirelessly transmits blood-pressure information through an airlink 16 to a wireless network 14. A host computer system 57 receives blood-pressure information from the wireless network 14 and avails it to a secondary computer system 69 for access by the patient or medical professional. The combination of the cuffless blood-pressure monitor 10 and the mobile device 15 allows a medical professional to continuously collect and monitor a patient's blood pressure, preferably for a short time period (e.g., 24 to 48 hours) during the patient's day-to-day activities. This approach avoids erroneous measurements due to ‘white coat syndrome’ and additionally means the patient's blood pressure can be monitored continuously, rather than during an isolated medical visit.

The cuffless blood pressure monitor 10 preferably features an optical finger-mounted module 13 that attaches to a patient's finger, and a wrist-mounted module 11 that attaches to the patient's wrist where a watch is typically worn. A cable 12 provides an electrical connection between the finger-mounted 13 and wrist-mounted 11 modules. During operation, the finger-mounted module 13 measures an optical ‘waveform’ that the blood-pressure monitor 10 processes to determine real-time beat-to-beat diastolic and systolic blood pressure, heart rate, and pulse oximetry. Methods for processing the optical waveform to determine blood pressure are described in the following co-pending patent applications, the entire contents of which are incorporated by reference: 1) U.S. patent application Ser. No. 10/810,237, filed Mar. 26, 2004, for a CUFFLESS BLOOD PRESSURE MONITOR AND ACCOMPANYING WEB SERVICES INTERFACE; 2) U.S. patent application Ser. No. 10/709,015, filed Apr. 7, 2004, CUFFLESS BLOOD-PRESSURE MONITOR AND ACCOMPANYING WIRELESS, INTERNET-BASED SYSTEM; 3) U.S. patent application Ser. No. 10/752,198, filed Jan. 6, 2004, for a WIRELESS, INTERNET-BASED MEDICAL DIAGNOSTIC SYSTEM; and co-pending U.S. Patent Application, filed Oct. 18, 2004, for a BLOOD PRESSURE MONITORING DEVICE FEATURING A CALIBRATION-BASED ANALYSIS.

A preferred mobile device 15 is based on Qualcomm's CDMA technology and features a chipset that integrates both hardware and software for the Bluetooth™ wireless protocol. These mobile devices 15 operate with the above-described blood-pressure monitor with little or no modifications. Such chipsets, for example, include the MSM family of mobile processors (e.g., MSM6025, MSM6050, and the MSM6500) and are described and compared in detail in http://www.qualcomm.com. For example, the MSM6025 and MSM6050 chipsets operate on both CDMA cellular and CDMA PCS wireless networks, while the MSM6500 operates on these networks and GSM wireless networks. In addition to circuit-switched voice calls, the wireless transmitters used in these chipsets transmit data in the form of packets at speeds up to 307 kbps in mobile environments. Those skilled in the pertinent art will recognize that mobile devices 15 with other chipsets may be utilized with the system 5 without departing from the scope and spirit of the present invention.

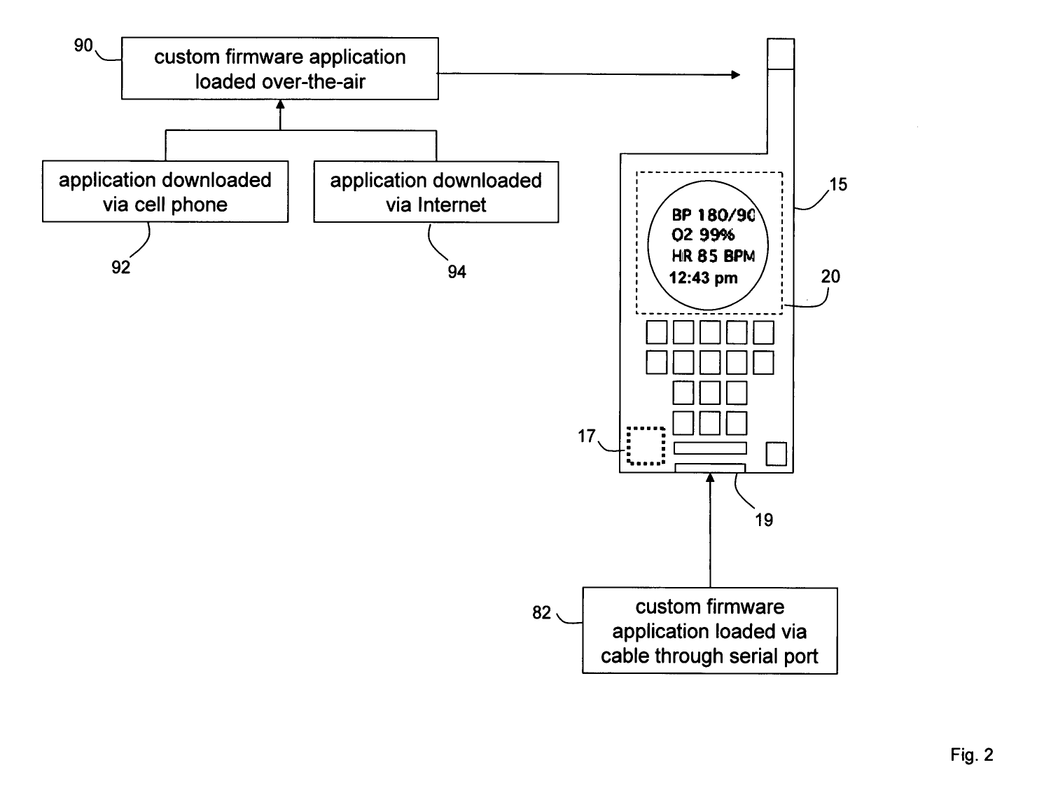

Referring to FIG. 2, the mobile device 15 supports a custom firmware application that displays and analyzes information from the blood-pressure monitor 10. The firmware application is written to operate on a variety of mobile device operating systems including BREW, Java, Pocket PC, Windows Mobile, Symbian, etc. At block 90, the custom firmware application is downloaded into the mobile device 15 using a wireless ‘over the air’ approach. Alternatively, at block 82, the custom firmware application is downloaded into the mobile device 15 using a ‘wired’ cable-based approach. For example, the mobile device 15 can contact a server that posts the firmware application. At block 92, using such an example, the application is selected and downloaded directly into the mobile device 15. Alternatively, at block 94, a user selects a firmware application using an Internet-accessible computer, which is downloaded to the mobile device 15. For the wired cable-based approach, the firmware application is loaded directly onto the mobile device 15 through a cable attached directly to the device's serial port 19. This approach, for example, is preferably used to download the firmware application to the device during a manufacturing process.

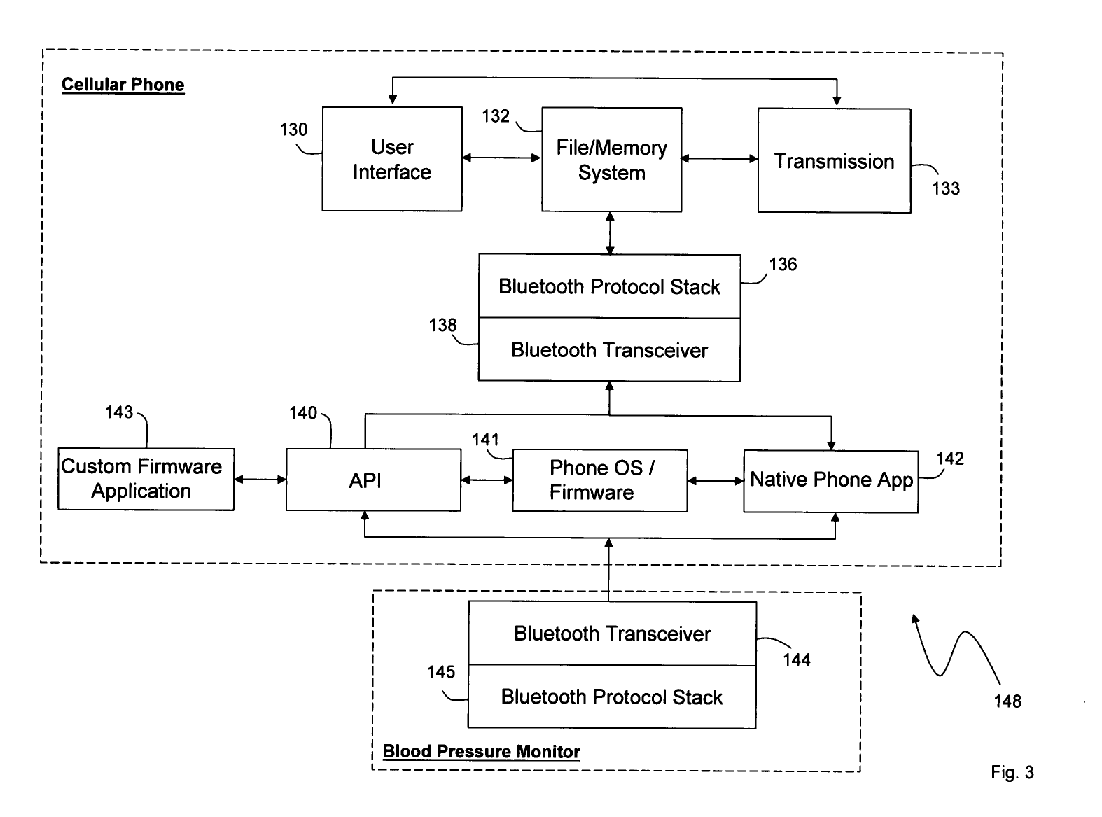

FIG. 3 shows a schematic drawing of a preferred embodiment of a firmware platform 148 operating on the mobile device 15. The firmware platform 148 supports a custom firmware application 143 that controls operations for receiving blood-pressure information from the blood-pressure monitor 10; processing, storing and displaying this information on the mobile device 15; and then transmitting the information through a wireless network 14. The custom firmware application 143 utilizes firmware functions integrated within an application-programming interface (API) 140 (e.g., BREW or Java APIs) that, in turn, communicate with a mobile device operating system/firmware 141 and a native phone firmware application 142. During operation, the custom firmware application 143 operates a firmware program that controls the device operating system/firmware 141 and native phone application 142 so that these systems collect information sent wirelessly preferably from a corresponding Bluetooth™ transceiver 144 in the blood-pressure monitor 10. The Bluetooth™ transceiver 144 within the monitor 10 uses a Bluetooth™ protocol stack 145 to send blood-pressure information to the mobile device 15. In a complimentary manner, the mobile device 15 uses a Bluetooth™ protocol stack 136 firmware layer to control its internal Bluetooth™ transceiver 138. Once blood-pressure information is sent from the monitor 10 to the mobile device 15, the custom firmware application 143 stores the blood-pressure information within a file memory system 132. At a later time a transmission firmware system 133 wirelessly transmits the information through a wireless network 14. Alternatively, the blood-pressure information is displayed on the device's user interface using a user-interface application 130 linked to and controlled by the custom firmware application 143.

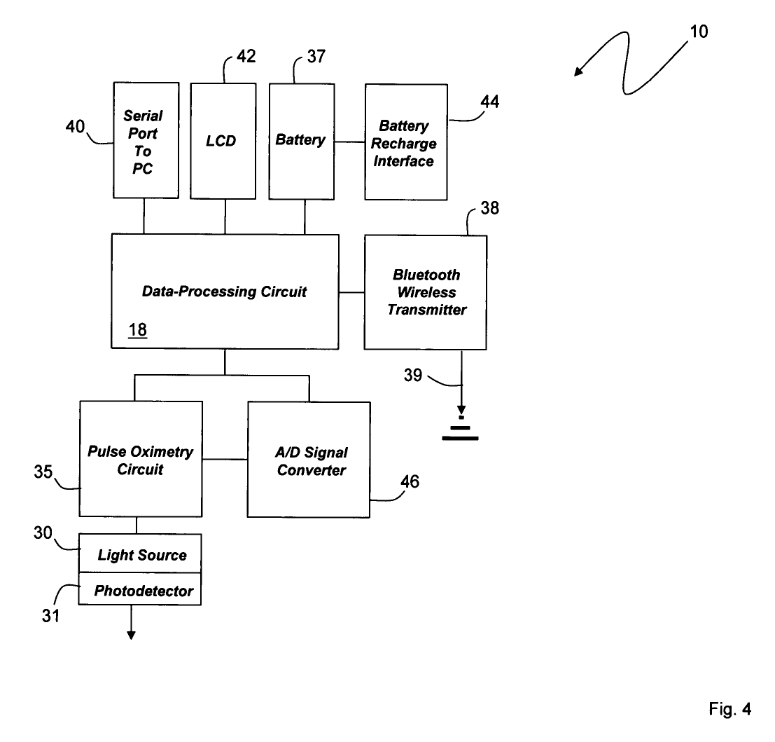

FIG. 4 shows a preferred embodiment of the electronic components featured in the blood-pressure monitor 10. A data-processing circuit 18 that implements the Bluetooth™ protocol stack 145 described with reference to FIG. 3 preferably controls the monitor 10. A Bluetooth™ wireless transceiver 38 sends information through an antenna 39 to a matched transceiver embedded within the mobile device 15. The monitor 10 can include a liquid crystal display (“LCD”) 42 that displays blood-pressure information for the user or patient. In another embodiment, the data-processing circuit 18 avails calculated information through a serial port 40 to an external personal computer, which then displays and analyzes the information using a client-side software application. A battery 37 powers all the electrical components within the monitoring device 10, and is preferably a metal hydride battery (generating 5-7V) that can be recharged through a battery-recharge interface 44.

To generate an optical waveform and measure blood pressure, pulse oximetry, and heart rate, the monitor 10 includes a light source 30 and a photodetector 31 embedded within the finger-mounted module shown in FIG. 1. The light source 30 typically includes light-emitting diodes that generate both red (λ˜630 nm) and infrared (λ˜900 nm) radiation. As the heart pumps blood through the patient's finger, blood cells absorb and transmit varying amounts of the red and infrared radiation depending on how much oxygen binds to the cells' hemoglobin. The photodetector 31 detects transmission at the red and infrared wavelengths, and in response generates a radiation-induced current that travels through a cable to the pulse-oximetry circuit 35 embedded within the wrist-worn module of FIG. 1. The pulse-oximetry circuit 35 connects to an analog-to-digital signal converter 46, which converts the radiation-induced current into a time-dependent optical waveform. The optical waveform is then sent back to the pulse-oximetry circuit 35 and data-processing circuit 18 and analyzed to determine the user's vital signs as described in this application and the above-mentioned co-pending patent applications, the contents of which have been incorporated by reference.

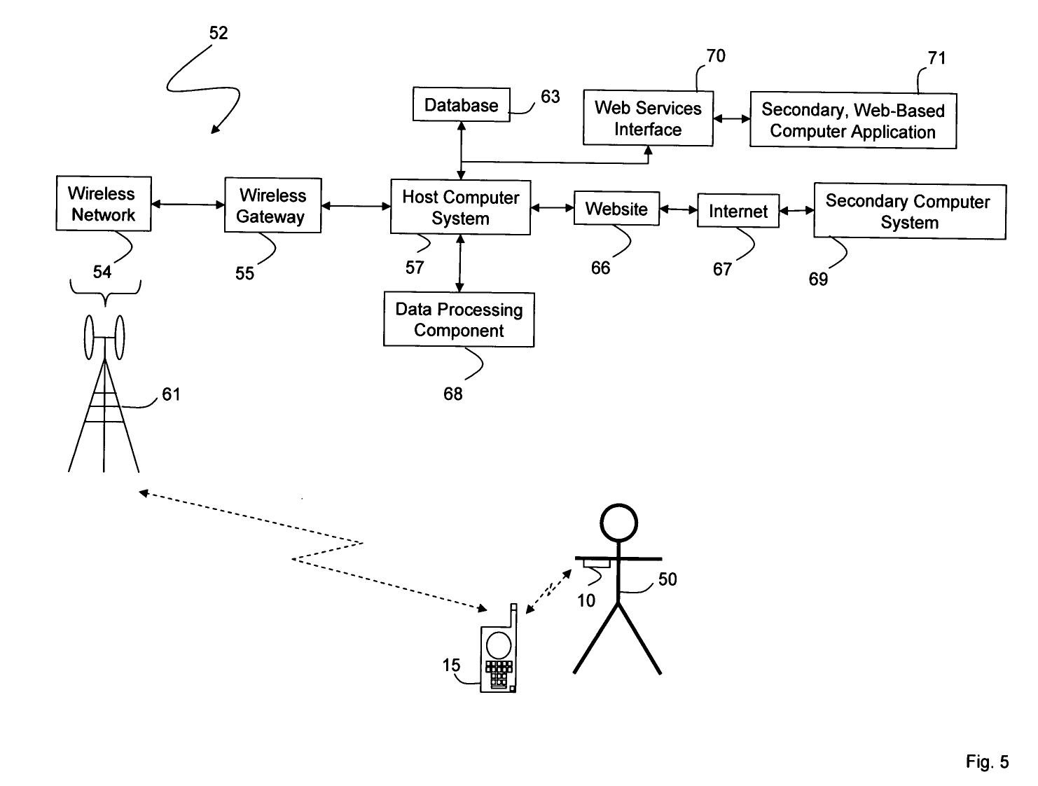

FIG. 5 shows a preferred embodiment of an Internet-based system 52 that operates in concert with the blood-pressure monitor 10 and mobile device 15 to send information from a patient 50 through a wireless network 54 to a web site 66 hosted on an Internet-based host computer system 57. A secondary computer system 69 accesses the website 66 through the Internet 67. The system 52 functions in a bi-directional manner, i.e. the mobile device 15 can both send and receive data. Most data flows from the mobile device 15; using the same network, however, the device can also receives data (e.g., ‘requests’ to measure data or text messages) and software upgrades as indicated in FIG. 2.

A wireless gateway 55 connects to the wireless network 54 and receives data from one or more mobile devices 15. The wireless gateway 55 additionally connects to a host computer system 57 that includes a database 63 and a data-processing component 68 for, respectively, storing and analyzing the data. The host computer system 57, for example, may include multiple computers, software pieces, and other signal-processing and switching equipment, such as routers and digital signal processors. The wireless gateway 55 preferably connects to the wireless network 54 using a TCP/IP-based connection, or with a dedicated, digital leased line (e.g., a frame-relay circuit or a digital line running an X.25 or other protocols). The host computer system 57 also hosts the web site 66 using conventional computer hardware (e.g. computer servers for both a database and the web site) and software (e.g., web server and database software).

During typical operation, the patient continuously wears the blood-pressure monitor 10 for a period of time, ranging from a 1-2 days to weeks. For longer-term monitoring (e.g. several months), the patient may wear the blood pressure monitor 10 for shorter periods of time during the day. To view information sent from the blood-pressure monitor 10, the patient or medical professional accesses a user interface hosted on the web site 66 through the Internet 67 from the secondary computer system 69. The system 52 may also include a call center, typically staffed with medical professionals such as doctors, nurses, or nurse practioners, whom access a care-provider interface hosted on the same website 66.

In an alternate embodiment, the host computer system 57 includes a web services interface 70 that sends information using an XML-based web services link to a secondary, web-based computer application 71. This application 71, for example, could be a data-management system operating at a hospital.

Many of the mobile devices 15 described above can be used to determine the patient's location using embedded position-location technology (e.g., GPS or network-assisted GPS). In situations requiring immediate medical assistance, the patient's location, along with relevant medical data collected by the blood pressure monitoring system, can be relayed to emergency response personnel.

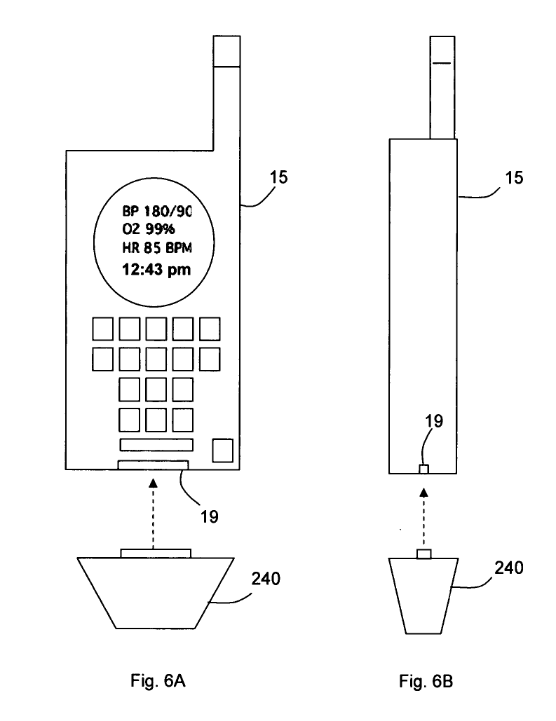

FIGS. 6A and 6B show an alternate embodiment of the invention wherein a removable, snap-on component 240 containing a wireless module (e.g., a module operating Bluetooth™, 802.11a, 802.11b, 802.1g, or 802.15.4 wireless protocols) connects to a serial port 19 located on a bottom portion the mobile device 15. This embodiment provides short-range wireless connectivity to mobile devices that lack built-it hardware for this capability. The serial port 19 supplies power, ground, and serial communication between the snap-on component 240 and the mobile device 15. Once connected, the snap-on component receives power and wirelessly communicates with the blood-pressure monitor 10 to send and receive information as described above.

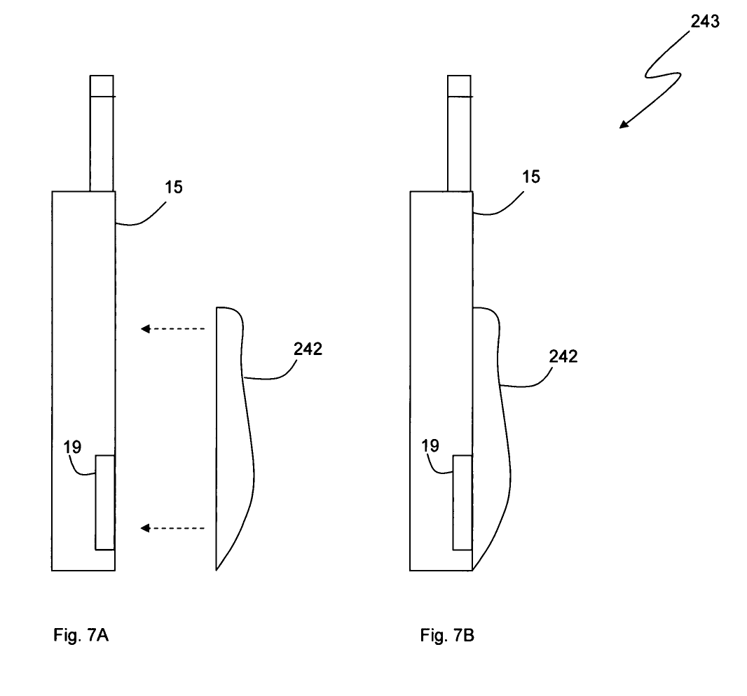

FIGS. 7A and 7B illustrate another alternative embodiment wherein a snap-on attachment 242, containing a wireless module similar to that described with reference to FIGS. 6A and 6B, connects to a serial port 19 located on a back portion of the mobile device 15 to provide short-range wireless connectivity as described above.

In other embodiments, the mobile device 15 described above is be replaced with a personal digital assistant (PDA) or laptop computer operating on a wireless network 14. In still other embodiments, the blood-pressure monitor 10 additionally includes a GPS module that receives GPS signals through an antenna from a constellation of GPS satellites and processes these signals to determine a location (e.g., latitude, longitude, and altitude) of the monitor 10 and, presumably, the patient. This location could be used to locate a patient during an emergency, e.g. to dispatch an ambulance. In still other embodiments, patient location information is obtained using position-location technology (e.g. network-assisted GPS) that is embedded in many mobile devices 15 that can be used for the blood-pressure monitoring system.

In other embodiments, the blood-pressure monitor 10 or the mobile device 15 use a ‘store and forward’ protocol wherein each device stores information when it is out of wireless coverage, and then transmits this information when it roams back into wireless coverage.

Still other embodiments are within the scope of the following claims.

1. A system for monitoring blood pressure, the system comprising:
a blood-pressure monitor comprising a measuring component that generates blood-pressure information and a first short-range wireless component configured to wirelessly transmit the blood-pressure information;
a mobile device comprising a chipset that includes: i) an embedded second short-range wireless component configured to receive the blood-pressure information from the first short-range wireless component; and ii) a long-range wireless component configured to transmit the blood-pressure information over a wireless network; and
a computer system configured to receive and display the blood-pressure information transmitted by the long-range wireless component.

2. The system of claim 1, wherein the first and second short-range wireless components operate a wireless protocol based on Bluetooth™, 802.11a, 802.11b, 802.1g, or 802.15.4.

3. The system of claim 1, wherein the mobile device is a cellular phone or a personal digital assistant.

4. The system of claim 1, wherein the chipset is configured to wirelessly transmit information over a terrestrial wireless network.

5. The system of claim 1, wherein the chipset comprises a microprocessor that supports a software application configured to receive information from the blood-pressure monitor.

6. The system of claim 5, wherein the software application is configured to display blood-pressure information on a display of the mobile device.

7. The system of claim 5, wherein the software application is configured to analyze the blood-pressure information.

8. The system of claim 6, wherein the software application is configured to graphically display the blood-pressure information.

9. The system of claim 5, wherein the software application is configured to store the blood-pressure information and transmit it at a later time.

10. The system of claim 9, wherein the software application is configured to transmit the blood-pressure information when the mobile device roams into wireless coverage.

11. The system of claim 1, wherein the measuring component comprises an optical system configured to measure blood pressure from a patient.

12. The system of claim 1, wherein the measurement component comprises a wrist-worn component and a finger-worn component.

13. The system of claim 1, wherein the measurement system is configured to measure blood pressure, heart rate, and pulse oximetry from a patient.

14. A system for monitoring vital signs, comprising:
a vital-sign monitor comprising a measuring component that generates vital-sign information and a first short-range wireless component configured to wirelessly transmit the vital-sign information;
a mobile device comprising a chipset that includes: i) an embedded second short-range wireless component configured to receive the vital-sign information from the first short-range wireless component; and ii) a long-range wireless component configured to transmit the vital-sign information over a wireless network; and
a computer system configured to receive and display the vital-sign information transmitted by the long-range wireless component.

15. A system for monitoring blood pressure, comprising:
a blood pressure monitor comprising a measuring component that generates blood-pressure information and a first short-range wireless component configured to wirelessly transmit the blood-pressure information;
a removable wireless component that connects to a serial port of a mobile device and comprises a second short-range wireless component configured to receive the blood-pressure information and send the blood-pressure information to the mobile device; and
a computer system configured to receive the blood-pressure information from the mobile device and display the blood-pressure information on an interface.

16. A method for monitoring a patient's real-time vital signs, the method comprising:
obtaining real-time vital sign measurements from a patient using a monitor attached to the patient;
wirelessly transmitting the real-time vital sign information from the monitor to a mobile device;
wirelessly transmitting the real-time vital sign information from the mobile device to a network;
receiving the real-time vital information over the network at a computer system; and
displaying the real-time vital sign information on the computer system.

17. The method of claim 16, wherein the real-time vital sign information is the patient's blood-pressure information.

18. The method of claim 16, wherein the real-time vital sign information is the patient's diastolic blood-pressure, systolic blood pressure, pulse oximetry and heart rate.

19. The method of claim 16, wherein the monitor comprises a first short-range wireless component that operates a wireless protocol based on Bluetooth™, 802.11a, 802.11b, 802.1g, or 802.15.4.

20. The method of claim 16, wherein the mobile device is a cellular phone or a personal digital assistant.

21. The method of claim 16, wherein the monitor comprises an optical system configured to measure blood pressure from a patient.

22. The method of claim 16, wherein the monitor comprises a wrist-worn component and a finger-worn component.

23. A system for monitoring blood pressure, the system comprising:
a blood-pressure monitor comprising a measuring component that generates blood-pressure information;
means for short-range wireless transmission of the blood-pressure information from the blood-pressure monitor;
a mobile device comprising means for receiving the blood-pressure information from the short-range wireless transmission means and means for long-range wireless transmission of the blood-pressure information over a wireless network; and
means to receive and display the blood-pressure information transmitted by the long-range wireless transmission means.
